(19) United States
(12) Patent Application Publication
(10) Pub. No.: US 2026/0191368 A1
(43) Pub. Date: Jul. 9, 2026

(54) Rotary Cleaning Device

(71) Applicant: Tian

(72) Inventor: Weiyue Yuan, Heyuan City (CN)

(21) Appl. No.: 19/009,666

(22) Filed: Jan. 3, 2025

(51) Int. Cl. A47K 7/04

(52) U.S. Cl. CPC A47K 7/04

(57) ABSTRACT

A rotary cleaning device includes a base, a liquid storage bottle, a liquid delivering structure, a rotating structure and a control circuit. The rotary cleaning device of the present invention is provided with a liquid storage bottle, the cleaning liquid can be directly stored inside the rotary cleaning device, and the user can conveniently perform cleaning process without the cumbersome operation of the external pipeline. At the same time, the rotary cleaning device is provided with a liquid delivering structure and a rotating structure to form an automatic mechanism of liquid delivering and rotation. In this way, the user only needs to start the rotary cleaning device, and the cleaning liquid can be automatically delivered to the part to be cleaned through the liquid delivering structure.

### FIELD OF THE INVENTION

The invention relates to the technical field of gynecological medical instruments, and in particular to a rotary cleaning device.

### BACKGROUND OF THE INVENTION

Gynecological irrigator is a medical device used to clean the female reproductive system by rinsing the area to be cleaned with cleaning liquid, so as to achieve the purpose of cleaning, preventing infection and promoting hygiene. Traditional gynecological irrigator is not equipped with cleaning liquid itself, it needs to be connected to a cleaning liquid source through external pipes and then delivered the cleaning liquid to the part to be cleaned. This design is mainly suitable for professional gynecological cleaning operations in hospitals or medical institutions. However, as women pay more attention to their own health management, more and more women are beginning to pay attention to the needs of self-cleaning and health care. Many women hope to be able to clean and care for themselves easily and safely at home for the sake of convenience and privacy. However, for ordinary users, the operation of connecting the cleaning liquid source to the gynecological irrigator through a pipe is relatively cumbersome, which can easily lead to liquid leakage and poor user experience when using the gynecological irrigator.

### SUMMARY OF THE DISCLOSURE

In view of this, the present invention proposes a rotary cleaning device, which aims to solve the problem that traditional gynecological irrigators need to be connected to pipelines when in use, and the operation is relatively cumbersome.

The present invention provides a rotary cleaning device, including a base, a liquid storage bottle, a liquid delivering structure, a rotating structure and a control circuit; wherein:
The liquid storage bottle comprises a cover body and a bottle body for containing cleaning liquid, the bottle body is provided below the base, the cover body is fixedly provided on the base, and the bottle body and the cover body are detachably connected;
The liquid delivering structure includes a liquid pump, a liquid inlet pipe, a liquid outlet pipe, a liquid outlet column and a sleeve, wherein the sleeve is fixedly provided on the base and is provided with a through hole, the liquid outlet column is provided above the sleeve, a hollow cavity is provided inside the liquid outlet column, a liquid outlet hole is provided on the outer wall of the liquid outlet column, and the hollow cavity is communicated with the liquid outlet hole; one end of the liquid inlet pipe is provided inside the bottle body, the other end of the liquid inlet pipe is connected to the liquid pump, one end of the liquid outlet pipe is connected to the liquid pump, and the other end of the liquid outlet pipe is connected to one end of the through hole;
The rotating structure includes a motor and a rotary cylinder, the bottom end of the rotary cylinder is fixedly connected to the output shaft of the motor, the top end of the rotary cylinder is fixedly connected to the liquid outlet column, the rotary cylinder is inserted into the sleeve and is provided concentrically with the sleeve, a hollow cylinder is provided inside the rotary cylinder, a liquid inlet groove is provided in the middle of the outer wall of the rotary cylinder, and the hollow cylinder is communicated with the liquid inlet groove and the hollow cavity respectively; the motor can drive the rotary cylinder to rotate relative to the sleeve through the output shaft, when the rotary cylinder rotates to the position where the liquid inlet groove is connected to the other end of the through hole, the cleaning liquid can flows into the hollow cylinder through the other end of the liquid outlet pipe, the through hole and the liquid inlet groove in sequence;
The control circuit includes a switch module, which is provided on the cover body and is electrically connected to the motor and the liquid pump respectively. When the motor and the liquid pump is controlled to start through the switch module of the control circuit, the motor drives the rotary cylinder to rotate through the output shaft, and then the rotary cylinder drives the liquid outlet column to rotate. When the rotary cylinder rotates to the position where the liquid inlet groove and the other end of the through hole are connected, the cleaning liquid in the bottle body flows through the liquid inlet pipe, the liquid pump, the liquid outlet pipe, the through hole, the liquid inlet groove, the hollow cylinder and the hollow cavity in sequence, and then the cleaning liquid is rotating sprays out through the liquid outlet hole.

In a possible implementation, the rotary cleaning device also includes an upper housing and a lower housing, the upper housing is provided above the sleeve, the outer wall of the upper housing is provided with a liquid outlet nozzle and a convex pattern, the liquid outlet column is fixedly provided inside the upper housing and is concentrically provided with the upper housing, the liquid outlet nozzle corresponds to the liquid outlet hole one by one and is connected to each other, the lower housing is fixedly provided below the base, and the installation positions of the motor and the liquid pump are separately provided upper and lower inside the lower housing.

In a possible implementation, the the convex pattern is provided in spiral.

In a possible implementation, the liquid outlet hole includes a first hole, a second hole, a third hole, a fourth hole and a fifth hole, the first hole is provided at the top of the liquid outlet column, the second hole and the third hole are staggered up and down on both sides of the liquid outlet column, the fourth hole and the fifth hole are staggered up and down on both sides of the liquid outlet column, the second hole is provided above the fourth hole, and the third hole is provided above the fifth hole.

In a possible implementation, a first sealing ring is provided at the contact interface between the cover body and the base, and a second sealing ring is provided at the contact interface between the cover body and the sleeve.

In a possible implementation, one end of the liquid inlet pipe is connected to a ball check valve.

In a possible implementation, the rotary cleaning device further includes an annular bearing, which is disposed between the rotary cylinder and the sleeve, and the annular bearing is provided on the upper part of the outer wall of the rotary cylinder and is concentrically disposed with the sleeve.

In a possible implementation, the liquid inlet groove includes a first groove and a second groove, and the first groove and the second groove are symmetrically and separated provided.

In a possible implementation, the liquid storage bottle is a transparent bottle, and scale lines are provided on the outer wall of the transparent bottle.

In a possible implementation, the control circuit further includes a battery, and the battery is disposed on the base and electrically connected to the switch module.

Compared with the prior art, the beneficial effects of the present invention are as follows: a rotary cleaning device includes a base, a liquid storage bottle, a liquid delivering structure, a rotating structure and a control circuit; wherein the liquid storage bottle includes a cover body and a bottle body for containing cleaning liquid, the liquid delivering structure includes a liquid pump, a liquid inlet pipe, a liquid outlet pipe, a liquid outlet column and a sleeve, the sleeve is fixedly provided on the base and is provided with a through hole, a hollow cavity is provided inside the liquid outlet column, a liquid outlet hole is provided on the outer wall of the liquid outlet column, the rotating structure includes a motor and a rotary cylinder; a hollow cylinder is provided inside the rotary cylinder, a liquid inlet groove is provided in the middle of the outer wall of the rotary cylinder, and the control circuit includes a switch module; when the motor and the liquid pump is controlled to start through the switch module of the control circuit, the motor drives the rotary cylinder to rotate through the output shaft, and then the rotary cylinder drives the liquid outlet column to rotate. When the rotary cylinder rotates to the position where the liquid inlet groove and the through hole are connected, the cleaning liquid in the bottle body flows through the liquid inlet pipe, the liquid pump, the liquid outlet pipe, the through hole, the liquid inlet groove, the hollow cylinder and the hollow cavity in sequence, and then the cleaning liquid is rotating sprays out through the liquid outlet hole. It can be seen that the rotary cleaning device of the present invention is provided with a liquid storage bottle, the cleaning liquid can be directly stored inside the rotary cleaning device, and the user can conveniently perform cleaning process without the cumbersome operation of the external pipeline. At the same time, the rotary cleaning device is provided with a liquid delivering structure and a rotating structure to form an automatic mechanism of liquid delivering and rotation. In this way, the user only needs to start the rotary cleaning device, and the cleaning liquid can be automatically delivered to the part to be cleaned through the liquid delivering structure. In addition, the rotating structure can also make the cleaning liquid spray out more evenly, ensuring that the sprayed cleaning liquid has good coverage and cleaning effect. This evenly spraying method not only improves the cleaning efficiency, but also effectively reduces the waste of cleaning liquid. The rotary cleaning device of the present invention is simple and convenient to use, users can easily carry and use whether at home or traveling, which is convenient for users to choose the time and place for cleaning, and improves the autonomy and convenience of women in personal hygiene management.

### BRIEF DESCRIPTION OF THE DRAWINGS

FIG. 1 is a schematic structural view of a rotary cleaning device provided by an embodiment of the present invention from the first angle.

FIG. 2 is a cross-sectional view of the plane A-A in FIG. 1.

FIG. 3 is a schematic structural view of a rotary cleaning device provided by an embodiment of the present invention from the second angle.

FIG. 4 is a exploded structural view of a rotary cleaning device provided by an embodiment of the present invention from the first angle.

FIG. 5 is a schematic structural view of a base, a liquid delivering structure, a rotating structure and a control circuit of a rotary cleaning device provided by an embodiment of the present invention.

FIG. 6 is a exploded structural view of a rotary cleaning device provided by an embodiment of the present invention from the second angle.

FIG. 7 is a schematic structural view of part A in FIG. 6.

### DETAILED DESCRIPTION OF THE PREFERRED EMBODIMENTS

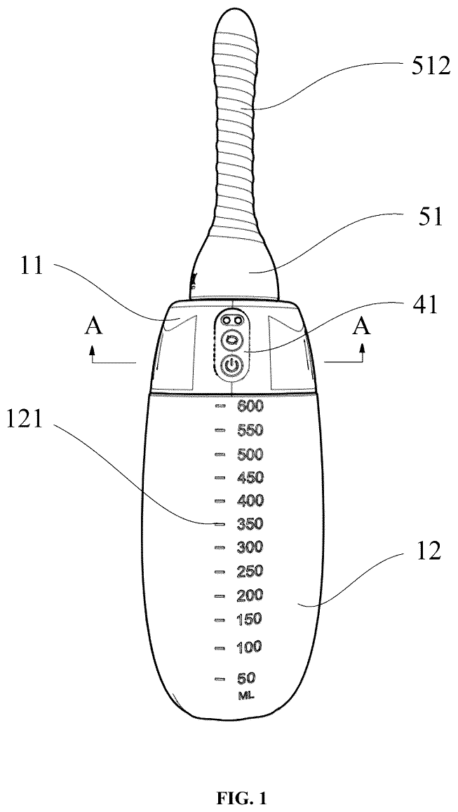

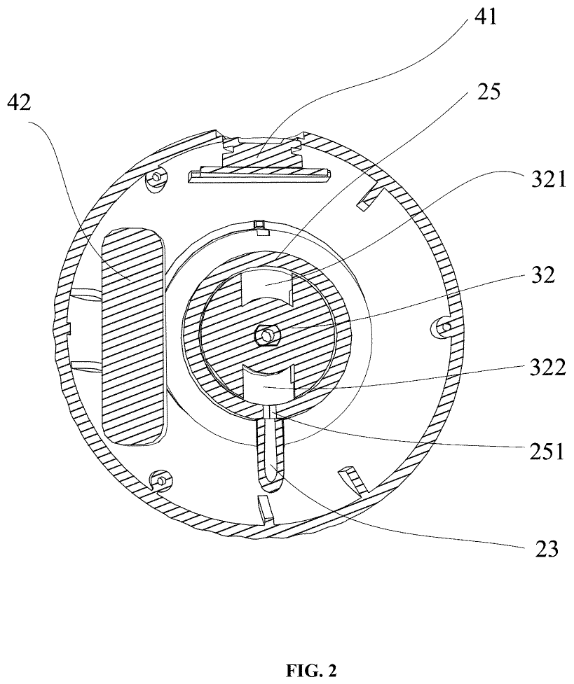

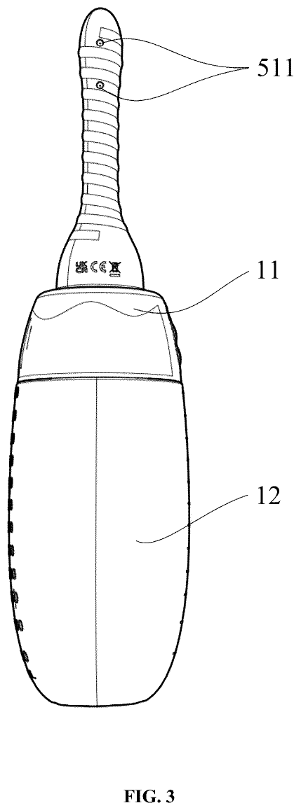

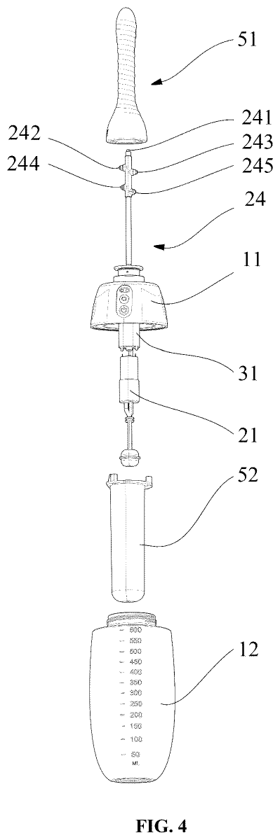

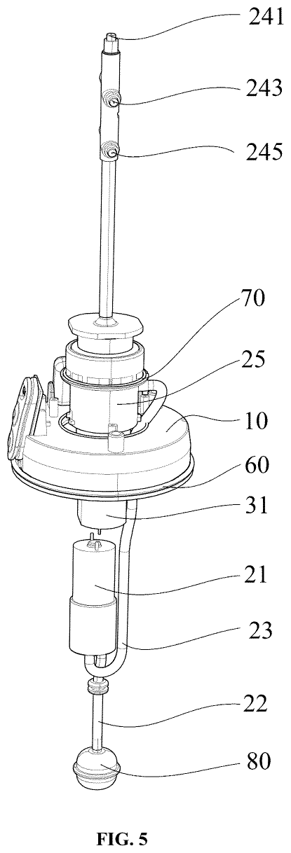

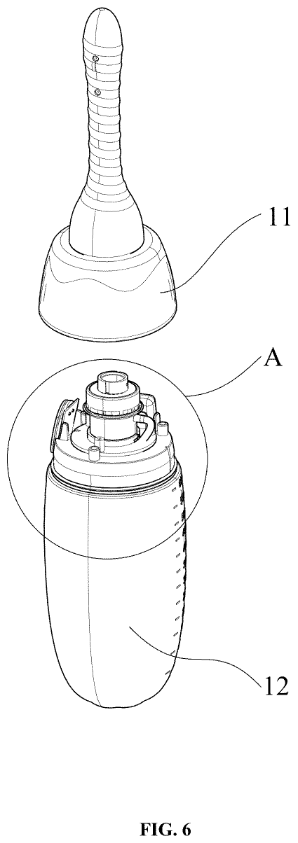

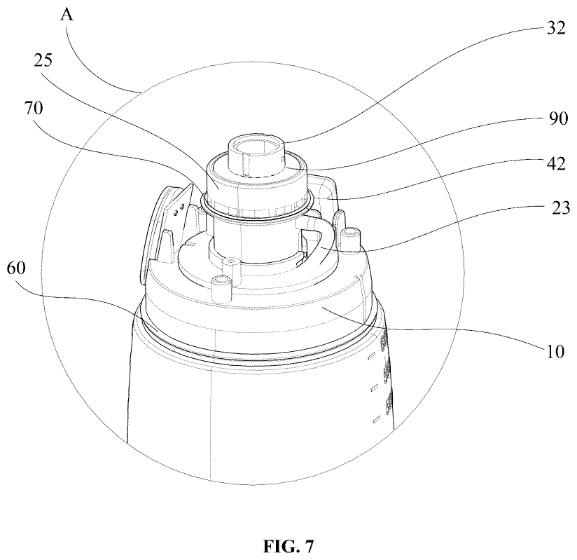

Other features, objects and advantages of the present invention will be more clearly and completely by the detailed description of the non-limiting embodiments with reference to the attached drawings (FIG. 1 to FIG. 7). Obviously, the described embodiments are only part of the embodiments of the present invention, but not all of them. Based on the embodiments of the present invention, all other embodiments obtained by ordinary technicians in the field without creative labor are within the scope of the present invention.

In the present invention, the use of orientation words such as “upper”, “lower” “bottom”, “back” only for the convenience of description, rather than indicating or implying the specific orientation, therefore it should not be construed as being limited to the description of the following embodiments.

In addition, it should be noted that the terms “first”, “second”, “third” and the like in the description and claims of the present invention and the above drawings are used to distinguish similar objects, which are not necessarily used to describe the specific order or sequence. It is to be understood that the data so used are interchangeable under appropriate circumstances in order to describe the embodiments of the invention herein.

Please refer to FIGS. 1-7, the present invention provides a rotary cleaning device, including a base 10, a liquid storage bottle, a liquid delivering structure, a rotating structure and a control circuit; wherein,

The liquid storage bottle includes a cover body 11 and a bottle body 12 for containing cleaning liquid, the bottle body 12 is provided below the base 10, the cover body 11 is fixedly provided on the base 10, and the bottle body 12 and the cover body 11 are detachably connected;

The liquid delivering structure includes a liquid pump 21, a liquid inlet pipe 22, a liquid outlet pipe 23, a liquid outlet column 24 and a sleeve 25, wherein the sleeve 25 is fixedly provided on the base 10 and is provided with a through hole 251, the liquid outlet column 24 is provided above the sleeve 25, a hollow cavity is provided inside the liquid outlet column 24, a liquid outlet hole is provided on the outer wall of the liquid outlet column 24, and the hollow cavity is communicated with the liquid outlet hole; one end of the liquid inlet pipe 22 is provided inside the bottle body 12, the other end of the liquid inlet pipe 22 is connected to the liquid pump 21, one end of the liquid outlet pipe 23 is connected to the liquid pump 21, and the other end of the liquid outlet pipe 23 is connected to one end of the through hole 251;

The rotating structure includes a motor 31 and a rotary cylinder 32, the bottom end of the rotary cylinder 32 is fixedly connected to the output shaft of the motor 31, the top end of the rotary cylinder 32 is fixedly connected to the liquid outlet column 24, the rotary cylinder 32 is inserted into the sleeve 25 and is provided concentrically with the sleeve 25, a hollow cylinder is provided inside the rotary cylinder 32, a liquid inlet groove is provided in the middle of the outer wall of the rotary cylinder 32, and the hollow cylinder is communicated with the liquid inlet groove and the hollow cavity respectively; the motor 31 can drive the rotary cylinder 32 to rotate relative to the sleeve 25 through the output shaft, when the rotary cylinder 32 rotates to the position where the liquid inlet groove is connected to the other end of the through hole 251, the cleaning liquid can flows into the hollow cylinder through the other end of the liquid outlet pipe 23, the through hole 251 and the liquid inlet groove in sequence;

The control circuit includes a switch module 41, which is provided on the cover body 11 and is electrically connected to the motor 31 and the liquid pump 21 respectively. When the motor 31 and the liquid pump 21 is controlled to start through the switch module 41 of the control circuit, the motor 31 drives the rotary cylinder 32 to rotate through the output shaft, and then the rotary cylinder 32 drives the liquid outlet column 24 to rotate. When the rotary cylinder 32 rotates to the position where the liquid inlet groove and the other end of the through hole 251 are connected, the cleaning liquid in the bottle body 12 flows through the liquid inlet pipe 22, the liquid pump 21, the liquid outlet pipe 23, the through hole 251, the liquid inlet groove, the hollow cylinder and the hollow cavity in sequence, and then the cleaning liquid is rotating sprays out through the liquid outlet hole.

Specifically, the rotary cleaning device can start the motor 31 and the liquid pump 21 at the same time to achieve the purpose of rotary rinsing. Of course, the user can also start the motor 31 separately to achieve the independent rotation of the rotary cylinder 32, so that the user can spray out the remaining cleaning liquid inside liquid delivering structure our of the the rotary cleaning device, thereby extending the service life of the rotary cleaning device. In addition, the user can also start the liquid pump 21 separately to achieve independent delivery of the cleaning liquid, which is convenient for centralized cleaning of specific parts, thereby significantly improving the cleaning efficiency. This flexible control method makes the rotary cleaning device more user-friendly during use, which can adapt to different scenarios, meet different user needs, and improves the overall user experience.

Specifically, the bottle body 12 and the cover body 11 are detachably connected, which can facilitate users to replace the cleaning liquid in time, increase the number of times the rotary cleaning device is used, and thus improve its practicality. The detachable connection method is provided in threaded connection, the user can simply tighten the bottle body 12 and the cover body 11 by rotating clockwise, and can easily loosened them by rotating counterclockwise. This design is not only easy to operate, but also ensures the stability of the connection, and effectively avoids loosening or leakage of the cleaning liquid during use. In addition, the water can be used as a source of cleaning liquid, but from the perspective of the health, it is not recommended to use directly for rinsing. The reason is because water may not be able to effectively remove bacteria and dirt, and lacks the necessary disinfection ingredients, besides, the tap water may also introduce impurities and chemicals, thereby increasing the risk of infection. In order to ensure the safety and effectiveness of cleaning, it is recommended to use specially formulated cleaning liquids, which usually contain mild disinfection ingredients and can better maintain the health and cleanliness of the female reproductive system. The bottle body 12 and the cover body 11 are designed to be detachably connected, the user can flexibly add water or other special cleaning liquid into the bottle body 12, so as to meet the different cleaning needs.

Compared with the prior art, the rotary cleaning device proposed in this embodiment is provided with a liquid storage bottle so that the cleaning liquid can be directly stored inside the rotary cleaning device. During use, the user can conveniently perform cleaning process without the cumbersome operation of the external pipeline. At the same time, the rotary cleaning device is provided with a liquid delivering structure and a rotating structure to form an automatic mechanism of liquid delivering and rotation. In this way, the user only needs to start the rotary cleaning device, and the cleaning liquid can be automatically delivered to the part to be cleaned through the liquid delivering structure. In addition, the rotating structure can also make the cleaning liquid spray out more evenly, ensuring that the sprayed cleaning liquid has good coverage and cleaning effect. This evenly spraying method not only improves the cleaning efficiency, but also effectively reduces the waste of cleaning liquid. The rotary cleaning device of the present invention is simple and convenient to use, users can easily carry and use whether at home or traveling, which is convenient for users to choose the time and place for cleaning, and improves the autonomy and convenience of women in personal hygiene management.

In some embodiments of the present application, the rotary cleaning device also includes an upper housing 51 and a lower housing 52, the upper housing 51 is provided above the sleeve 25, the outer wall of the upper housing 51 is provided with a liquid outlet nozzle 511 and a convex pattern 512, the liquid outlet column 24 is fixedly provided inside the upper housing 51 and is concentrically provided with the upper housing 51, the liquid outlet nozzle 511 corresponds to the liquid outlet hole one by one and is connected to each other, the lower housing 52 is fixedly provided below the base 10, and the installation positions of the motor 31 and the liquid pump 21 are separately provided upper and lower inside the lower housing 52.

Specifically, the upper housing 51 is smooth processed to ensure that it will not cause any scratches or discomfort to the user during use. The lower housing 52 is provided inside the bottle body 12 and is sealing connected to the base 10. The motor 31 and the liquid pump 21 are installed in the corresponding installation positions inside the lower housing 52, which can effectively prevent the cleaning liquid from eroding the motor 31 and the liquid pump 21. Generally speaking, the installation position of the motor 31 is provided above the installation position of the liquid pump 21 to facilitate the connection between the motor 31 and the rotary cylinder 32, thereby improving the compactness of the structure of the rotary cleaning device. The installation position of the motor 31 is separated from the installation position of the liquid pump 21, which can effectively reduce the impact of the vibration generated during the operation of motor 31 on the liquid pump 21, and also reduce the interference of the vibration of the liquid pump 21 itself on other components. This separation design can improve the overall performance and reliability of the rotary cleaning device.

in some embodiments of the present application, the convex pattern 512 is provided in spiral.

Specifically, the spiral convex pattern 512 can increase the contact area between the upper housing 51 and the part to be cleaned, thereby increasing the action range of the cleaning liquid and making the cleaning more thorough. During the cleaning process, based on the spiral structure of the convex pattern 512, the cleaning liquid can more effectively wash away the dirt when flowing, reducing the possibility of dirt residue on the surface of the part to be cleaned. At the same time, when a foreign object is stuck in the part to be cleaned, the spiral convex pattern 512 can assist in the flow of the cleaning liquid, and smoothly take out the foreign object and avoid foreign object residue with its special shape and structure. In addition, the control circuit can also control the motor 31, which drives the upper housing 51 rotates synchronously with the liquid outlet column 24 in a forward or reverse direction, so as to further enhance the cleaning effect.

In some embodiments of the present application, the liquid outlet hole includes a first hole 241, a second hole 242, a third hole 243, a fourth hole 244 and a fifth hole 245, the first hole 241 is provided at the top of the liquid outlet column 24, the second hole 242 and the third hole 243 are staggered up and down on both sides of the liquid outlet column 24, the fourth hole 244 and the fifth hole 245 are staggered up and down on both sides of the liquid outlet column 24, the second hole 242 is provided above the fourth hole 244, and the third hole 243 is provided above the fifth hole 245.

Specifically, in order to ensure the sufficient pressure and flow volume of the sprayed cleaning liquid, the hollow cavity of the liquid outlet column 24 is usually thinner, so that the cleaning liquid can be quickly sprayed out through the liquid outlet hole, preventing the cleaning liquid from accumulating inside the liquid outlet column 24 and reducing retention. By arranging the first hole 241 at the top of the liquid outlet column 24, it can be ensured that the cleaning liquid is sprayed out at a forward spraying angle, thereby covering a wider cleaning area and improving the cleaning effect. The second hole 242, the third hole 243, the fourth hole 244 and the fifth hole 245 form a arrangement mode in which the liquid outlet holes are staggered up and down, which can make the cleaning liquid spraying more evenly and avoid concentrated spraying on a certain position, thereby improving the comprehensiveness of cleaning.

In some embodiments of the present application, a first sealing ring 60 is provided at the contact interface between the cover body 11 and the base 10, and a second sealing ring 70 is provided at the contact interface between the cover body 11 and the sleeve 25.

Specifically, by providing the first sealing ring 60 and the second sealing ring 70, the cleaning liquid can be effectively prevented from flowing through the control circuit and other components covered by the cover 11 during use, avoiding the cleaning liquid from contaminating them, and further extending the service life of the rotary cleaning device.

In some embodiments of the present application, one end of the liquid inlet pipe 22 is connected to a ball check valve 80.

Specifically, the ball check valve 80 can effectively prevent the cleaning liquid from flowing back into the liquid inlet pipe 22 when the rotary cleaning device stops working, ensuring that the cleaning liquid always maintains a predetermined flow direction to avoid confusion. In addition to preventing the cleaning liquid from flowing back, the ball check valve 80 can also ensure that the cleaning liquid quickly flows into the liquid inlet pipe 22 when the user needs perform cleaning, thereby improving the cleaning efficiency of the rotary cleaning device.

In some embodiments of the present application, the rotary cleaning device further includes an annular bearing 90, which is disposed between the rotary cylinder 32 and the sleeve 25, and the annular bearing 90 is provided on the upper part of the outer wall of the rotary cylinder 32 and is concentrically disposed with the sleeve 25.

Specifically, the annular bearing 90 is provided concentrically with the rotary cylinder 32 and the sleeve 25, which can ensure that the rotary cylinder 32 is evenly stressed and evenly supported during the rotation process, thereby enhancing the stability of the rotary cylinder 32 and reducing shaking and wear during the rotation process, and ensuring that the rotary cleaning device runs more smoothly. The design of the annular bearing 90 reduces the friction between the rotary cylinder 32 and the sleeve 25, facilitating the rotary cylinder 32 to rotate more smoothly.

In some embodiments of the present application, the liquid inlet groove includes a first groove 321 and a second groove 322, and the first groove 321 and the second groove 322 are symmetrically and separated provided.

Specifically, when the rotary cylinder 32 rotates to the position where the liquid inlet groove is connected to the through hole 251, the cleaning liquid can flows into the hollow cylinder through the liquid inlet groove. On the contrary, when the rotary cylinder 32 rotates to the position where the liquid inlet groove is separated from the through hole 251, the cleaning liquid cannot enter the hollow cylinder through the liquid inlet groove. The liquid inlet groove includes two symmetrical and separated grooves, compared with traditional one groove, it can increase the probability of the cleaning liquid entering the hollow cylinder and ensure that a sufficient flow of cleaning liquid is sprayed out during the cleaning process. The first groove 321 and the second groove 322 are both provided in the middle of the outer wall of the rotary cylinder 32, and will not affect the stable connection between the bottom end of the rotary cylinder 32 and the output shaft of the motor 31.

In some embodiments of the present application, the liquid storage bottle is a transparent bottle, and the scale lines 121 are provided on the outer wall of the transparent bottle.

Specifically, the transparent bottle includes a transparent glass bottle and a transparent plastic bottle. The design of the transparent bottle allows the user to visually observe the liquid level of the cleaning liquid in the bottle body 12, which is convenient for monitoring the remaining volume of the cleaning liquid at any time, and avoiding affecting the cleaning effect due to insufficient cleaning liquid. By providing scale lines 121 on the transparent bottle, the user can accurately control the volume of cleaning liquid to meet different cleaning needs and usage scenario requirements. It should be noted that the scale lines 121 on the transparent bottle can be provided as two sets, in order to facilitate the user to observe the liquid level of the cleaning liquid in the bottle body 12 from different angles, ensuring that the liquid level can be clearly read in various usage environments. In this embodiment, the maximum capacity of the bottle body 12 is 600 ml, and the scale lines 121 are spaced at 50 ml, with a total of twelve scale lines 121. The large-capacity design of 600 ml allows the user to store more cleaning liquid at one time when performing cleaning operations, reducing the cumbersome steps of adding cleaning liquid frequently, and further improving the cleaning efficiency.

In some embodiments of the present application, the control circuit further includes a battery 42, and the battery 42 is disposed on the base 10 and electrically connected to the switch module 41.

Specifically, the battery 42 can be a rechargeable battery, in order to facilitate the repeated use of the rotary cleaning device and reduce the cleaning cost of the user. The user only needs a simple charging operation to recharge the power of the rotary cleaning device at any time, avoiding cleaning interruptions due to insufficient power, and improving the convenience of use.

The rotary cleaning device provided in this embodiment is easy and flexible to use, which can meet the needs of female users for efficient cleaning, and has has good market prospects and application value.

Hereinafter, embodiments of the present invention have been described in detail with reference to the accompanying drawings. While the description above refers to the particular embodiments of the present invention, it will be understood that many modifications may be made without departing from the spirit thereof. Any equivalent replacement or modification would fall within the protection scope of the present invention.

What is claimed is:

1. A rotary cleaning device, characterized in that it includes a base, a liquid storage bottle, a liquid delivering structure, a rotating structure and a control circuit;
wherein the liquid storage bottle comprises a cover body and a bottle body for containing cleaning liquid, the bottle body is provided below the base, the cover body is fixedly provided on the base, and the bottle body and the cover body are detachably connected;
wherein the liquid delivering structure includes a liquid pump, a liquid inlet pipe, a liquid outlet pipe, a liquid outlet column and a sleeve, wherein the sleeve is fixedly provided on the base and is provided with a through hole, the liquid outlet column is provided above the sleeve, a hollow cavity is provided inside the liquid outlet column, a liquid outlet hole is provided on the outer wall of the liquid outlet column, and the hollow cavity is communicated with the liquid outlet hole; one end of the liquid inlet pipe is provided inside the bottle body, the other end of the liquid inlet pipe is connected to the liquid pump, one end of the liquid outlet pipe is connected to the liquid pump, and the other end of the liquid outlet pipe is connected to one end of the through hole;
wherein the rotating structure includes a motor and a rotary cylinder, the bottom end of the rotary cylinder is fixedly connected to the output shaft of the motor, the top end of the rotary cylinder is fixedly connected to the liquid outlet column, the rotary cylinder is inserted into the sleeve and is provided concentrically with the sleeve, a hollow cylinder is provided inside the rotary cylinder, a liquid inlet groove is provided in the middle of the outer wall of the rotary cylinder, and the hollow cylinder is communicated with the liquid inlet groove and the hollow cavity respectively; the motor can drive the rotary cylinder to rotate relative to the sleeve through the output shaft, when the rotary cylinder rotates to the position where the liquid inlet groove is connected to the other end of the through hole, the cleaning liquid can flows into the hollow cylinder through the other end of the liquid outlet pipe, the through hole and the liquid inlet groove in sequence;
wherein the control circuit includes a switch module, which is provided on the cover body and is electrically connected to the motor and the liquid pump respectively. When the motor and the liquid pump is controlled to start through the switch module of the control circuit, the motor drives the rotary cylinder to rotate through the output shaft, and then the rotary cylinder drives the liquid outlet column to rotate, and when the rotary cylinder rotates to the position where the liquid inlet groove and the other end of the through hole are connected, the cleaning liquid in the bottle body flows through the liquid inlet pipe, the liquid pump, the liquid outlet pipe, the through hole, the liquid inlet groove, the hollow cylinder and the hollow cavity in sequence, and then the cleaning liquid is rotating sprays out through the liquid outlet hole.

2. The rotary cleaning device according to claim 1, wherein the rotary cleaning device also includes an upper housing and a lower housing, the upper housing is provided above the sleeve, the outer wall of the upper housing is provided with a liquid outlet nozzle and a convex pattern, the liquid outlet column is fixedly provided inside the upper housing and is concentrically provided with the upper housing, the liquid outlet nozzle corresponds to the liquid outlet hole one by one and is connected to each other, the lower housing is fixedly provided below the base, and the installation positions of the motor and the liquid pump are separately provided upper and lower inside the lower housing.

3. The rotary cleaning device according to claim 2, wherein the convex pattern is provided in spiral.

4. The rotary cleaning device according to claim 2, wherein the liquid outlet hole includes a first hole, a second hole, a third hole, a fourth hole and a fifth hole, the first hole is provided at the top of the liquid outlet column, the second hole and the third hole are staggered up and down on both sides of the liquid outlet column, the fourth hole and the fifth hole are staggered up and down on both sides of the liquid outlet column, the second hole is provided above the fourth hole, and the third hole is provided above the fifth hole.

5. The rotary cleaning device according to claim 1, wherein a first sealing ring is provided at the contact interface between the cover body and the base, and a second sealing ring is provided at the contact interface between the cover body and the sleeve.

6. The rotary cleaning device according to claim 1, wherein one end of the liquid inlet pipe is connected to a ball check valve.

7. The rotary cleaning device according to claim 1, wherein the rotary cleaning device further includes an annular bearing, which is disposed between the rotary cylinder and the sleeve, and the annular bearing is provided on the upper part of the outer wall of the rotary cylinder and is concentrically disposed with the sleeve.

8. The rotary cleaning device according to claim 1, wherein the liquid inlet groove includes a first groove and a second groove, and the first groove and the second groove are symmetrically and separated provided.

9. The rotary cleaning device according to claim 1, wherein the liquid storage bottle is a transparent bottle, and scale lines are provided on the outer wall of the transparent bottle.

10. The rotary cleaning device according to claim 1, wherein the control circuit further includes a battery, and the battery is disposed on the base and electrically connected to the switch module.
